(19) United States
(12) Patent Application Publication
(10) Pub. No.: US 2020/0088732 A1
(43) Pub. Date: Mar. 19, 2020

(54) METHODS FOR THE DIAGNOSIS AND TREATMENT OF PANCREATIC DUCTAL ADENOCARCINOMA

(71) Applicants: INSERM (Institut National de la Santé et de la Recherche Mèdicale), Paris (FR); Université d'Aix Marseille, Marseille Cedex 07 (FR); Institut Jean Paoli & Irene Calmettes, Marseille (FR); Centre National de la Recherche Scientifique (CNRS), Paris (FR)

(72) Inventors: Richard TOMASINI, Marseille Cedex 09 (FR); Jérémy NIGRI, Marseille Cedex 09 (FR)

(21) Appl. No.: 16/604,844

(22) PCT Filed: Apr. 12, 2018
(86) PCT No.: PCT/EP2018/059462

§ 371 Date: Oct. 11, 2019

(30) Foreign Application Priority Data
Apr. 13, 2017 (EP) 17305439.6

(51) Int. Cl. G01N 33/574; A61K 45/06; A61K 31/517; A61K 38/17; A61K 31/4985; A61K 31/277

(52) U.S. Cl. CPC G01N 33/57438; A61K 45/06; A61K 31/517; G01N 2500/04; A61K 31/4985; A61K 31/277; A61K 38/1774

(57) ABSTRACT

The invention relates to methods for predicting the survival time of patients suffering from pancreatic ductal adenocarcinoma (PDA) and methods for the treatment of PDA. The inventors investigated the role of Pancreatitis Associated Protein (PAP/REG3A) as prognostic marker associated with PDA, evaluated its implication in PDA associated nervous system alterations (perineural invasion (PNI)) and its impact on PDAs' patients survival. Using an exvivo assay, they determined its influence on PNI and correlated it with the prognostic value of PAP/REG3A as a circulating biomarker. They demonstrated that PAP/REG3A enhance cancer cells migration and invasion abilities. By activating JAK/STAT signaling pathways, PAP/REG3A favors PNI, known to be associated with relapse after surgery. They also analyzed the level of PAP/REG3A in serum from healthy donors or patients with PDA from three different cohorts and demonstrate that PAP/REG3A is a biomarker of shorter survival as well as poor surgical outcomes with reduced disease-free survival. Thus, the invention relates to a method for predicting the survival time of a patient suffering from PDA and REG3A inhibitors for use in the treatment of PDA.

### FIELD OF THE INVENTION

The present invention relates to methods for predicting the survival time of patients suffering from pancreatic ductal adenocarcinoma (PDA). The present invention also relates to methods and pharmaceutical compositions for the treatment of PDA.

### BACKGROUND OF THE INVENTION

Pancreatic ductal adenocarcinoma (PDA) is the fourth leading cause of cancer death and is expected to become second in rank by 2030 [1]. It is among the most lethal of all cancers, with a 5-year survival rate of only 5% [1]. Because of its aggressiveness and the absence of symptoms, most of patients are diagnosed at an advanced stage, often metastatic, limiting their access to surgery. Palliative treatments have a reduced efficiency, even for recent combinatory treatments as Folfirinox or gemcitabine plus nab-paclitaxel [2,3] which improve global survival of 3-5 months but restricted to patients meeting several global health criteria ensuring their likehood to withstand important secondary effects. At present, numerous studies intend to open new therapeutic options integrating the impact of non-cancerous cells (stroma or intra-tumoral microenvironment) [4,5] or targeting PDA-associated hallmarks [6,7]. Among those, deciphering the drastic modulations of the nervous system compartment in PDA constitutes a potent source of biomarkers and therapeutic targets that could improve survival and quality of life.

Clinically reported for decades, the alterations of nervous system in pancreatic cancer include figures of neural remodeling (NR) as well as peri-neural invasion (PNI) [8]. While NR is characterized by an increased nerve size and density due to peripheral nerve fibers infiltration and axonogenesis [8], PNI refers to the presence of cancer cells within the perineurium or the endoneurium of nerve fibers. NR and PNI are associated with worst prognosis, shorten survival, recurrence and also linked with local or distant dissemination as well as neuropathic pain [9]. Such clinical associations open interesting therapeutic windows providing that the molecular basis of such profound and affecting alterations, at present unknown, could be uncovered. Interestingly, the importance of the nervous system in initiation and progression of PDA was recently confirmed, creating a link between inflammation and Kras-induced neoplasia [10]. Indeed, the consistent presence of inflammatory environment in pre-malignant lesions of PDA as well as in PDA itself foster niches where inflammatory mediators impact both cancer cells and nervous system.

One of the main PDAs' hallmarks is the massive presence of non-tumor cells [11], mainly immune cells and cancer associated fibroblasts (CAFs) [12,13]. Interestingly, those cells are widely reported as “factories”, producing tremendous amount of secreted factors impacting either cancer [5,14] and nerve cells [15]. Among those mediators, pro-inflammatory cytokines were correlated to clinicopathologic parameters, chemoresistance and survival [16,17]. Recently, an inflammatory gene signature was also reported in CAFs following chemotherapy treatment, suggesting a drastic impact of CAFs in pro-inflammatory response following treatment and the consequent impact on patients' survival [18]. Interestingly, such molecules were also depicted as drivers of PDAs associated neuronal plasticity [8] reinforcing the need to improve our knowledge on the link between inflammation, nervous system alterations and PDA.

In the present invention, the inventors evaluated the role of PAP/REG3A as a prognostic marker associated with clinicopathologic features of PDA. Human PAP/REG3A (mouse PAP/REG3β), or Pancreatitis-Associated Protein, is a C-type lectin-like secreted protein discovered for its implication during pancreatic diseases as acute pancreatitis [19], diabetes [20] or cystic fibrosis [21]. More recently, the tumor promoter role of PAP/REG3A in PDA was assessed through its implication in M1/M2 macrophages polarization [22] and tumor cell growth under IL6-associated inflammatory conditions [23]. A suspected role of PAP/REG3A as biomarker for PDA was reported but still remains unclear [24].

Regarding the roles of PAP/REG3A mentioned above as well as its implication in nervous system [25,26], the inventors evaluated its implication in PDA associated nervous system alterations, and more specifically in PNI, as well as its potential as a prognostic marker. Here, the inventors further examined the impact of peri-tumoral microenvironment, through PAP/REG3A secretion, and aimed at unraveling its impact on PDAs' patients survival. Using a new ex-vivo assay, the inventors determined its influence on PNI and correlated it with the prognostic value of PAP/REG3A as a circulating biomarker in order to better stratify PDA patients.

### SUMMARY OF THE INVENTION

The present invention relates to methods for predicting the survival time of patients suffering from pancreatic ductal adenocarcinoma (PDA). The present invention also relates to methods and pharmaceutical compositions for the treatment of PDA.

### DETAILED DESCRIPTION OF THE INVENTION

The inventors investigated the role of Pancreatitis Associated Protein (PAP/REG3A) as prognostic marker associated with clinicopathologic features of pancreatic ductal adenocarcinoma (PDA). The inventors also evaluated its implication in PDA associated nervous system alterations, and more specifically in peri-neural invasion (PNI), as well its potential as prognostic marker. The inventors further examined the impact of peri-tumoral microenvironment, through PAP/REG3A secretion, and its impact on PDAs' patients survival. Using an ex-vivo assay, the inventors determined its influence on PNI and correlated it with the prognostic value of PAP/REG3A as a circulating biomarker in order to better stratify PDA patients. The inventors demonstrated that PAP/REG3A is produced in PDA by inflamed acinar cells from the peri-tumoral microenvironment then enhance cancer cells migration and invasion abilities. More specifically, using peri-neural ex-vivo assays, the inventors revealed that PAP/REG3A, by activating JAK/STAT signaling pathways in cancer cells, favors peri-neural invasion, known to be associated with relapse after surgery. The inventors also analyzed the level of PAP/REG3A in serum from healthy donors or patients with PDA from three different cohorts. An optimal PAP/REG3A cut-off value of 17.5 μg/mL was identified; patients with baseline PAP/REG3A levels of 17.5 μg/mL or higher had shorter survival as well as poor surgical outcomes with reduced disease-free survival. Altogether, the inventors demonstrated that PAP/REG3A is a promising biomarker for monitoring pancreatic cancer prognosis and that therapeutic targeting of PAP/REG3A activity in PDA limits tumor cell aggressiveness and peri-neural invasion.

Accordingly the first object of the present invention relates to a method for predicting the survival time of a patient suffering from pancreatic ductal adenocarcinoma (PDA) comprising the steps of: i) determining the expression level of REG3A in a biological sample obtained from the patient, ii) comparing the expression level determined at step i) with a predetermined reference value and iii) concluding that the patient will have a long survival time when the level determined at step i) is lower than the predetermined reference value or concluding that the patient will have a short survival time when the level determined at step i) is equal or higher than the predetermined reference value.

As used herein, the term “patient” denotes a mammal. Typically, a patient according to the invention refers to any patient (preferably human) afflicted with pancreatic ductal adenocarcinoma (PDA). The term “patient” also refers to a PDA resected patient, a patient suffering from pancreatic ductal adenocarcinoma (PDA) following surgical PDA resection.

As used herein, the term “pancreatic ductal adenocarcinoma” or “PDA” has its general meaning in the art and refers to pancreatic ductal adenocarcinoma such as revised in the World Health Organisation Classification C25. The term “pancreatic ductal adenocarcinoma” also refers to metastatic pancreatic cancer, exocrine pancreatic cancer, locally advanced PDAC and PDA associated neural remodelling (PANR).

The term “PDA associated neural remodeling” or “PANR” has its general meaning in the art and refers to conditions resulting in higher nerve densities in PDA due to peripheral nerve fibers infiltration and axonogenesis (Ceyhan et al., 2009; Stopczynski et al., 2014). The term “PDA associated neural remodeling” also refers to alterations caused by the PDA intratumoral microenvironment (Secq et al., 2015), this includes increased neural density, hypertrophy and pancreatic neuritis, as well as intra and extrapancreatic perineural invasion (PNI) by cancer cells (Bapat et al., 2011; Ceyhan et al., 2009). The term “PDA associated neural remodeling” also refers to neural remodelling which is clinically correlated with neuropathic pain (Bapat et al., 2011).

The term “biological sample” refers to any biological sample derived from the patient such as blood sample, plasma sample, serum sample or PDA sample.

As used herein, the term “REG3A” has its general meaning in the art and refers to Regenerating gene protein (REG) 3A or Regenerating islet-derived protein 3-alpha, a secretory pancreas protein with pro-growth function. The term “REG3A” also refers to REG3A, also named as pancreatic associated protein (PAP) or the encoded protein of genes expressed in heptocarcinoma-intestine-pancreas (HIP), a member of the REG family (REG1A, REG1B, REG3A, REG4) (Liu et al., 2015; Wang et al., 2014).

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. In general, OS rates do not specify whether cancer survivors are still undergoing treatment at five years or if they've become cancer-free (achieved remission). DFS gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

In some embodiment, the method of the invention in performed for predicting the overall survival (OS), progression-free survival (PFS) and/or the disease-free survival (DFS) of a patient suffering from pancreatic ductal adenocarcinoma (PDA) following PDA resection.

In some embodiments, the present invention relates to a method for predicting the overall survival (OS) of a PDA resected patient comprising the steps of: i) determining the expression level of REG3A in a biological sample obtained from the patient, ii) comparing the expression level determined at step i) with a predetermined reference value and iii) concluding that the patient will have a long survival time when the level determined at step i) is lower than the predetermined reference value or concluding that the patient will have a short survival time when the level determined at step i) is equal or higher than the predetermined reference value.

In some embodiments, the present invention relates to a method for predicting the disease-free survival (DFS) of a PDA resected patient comprising the steps of: i) determining the expression level of REG3A in a biological sample obtained from the patient, ii) comparing the expression level determined at step i) with a predetermined reference value and iii) concluding that the patient will have a long disease-free survival when the level determined at step i) is lower than the predetermined reference value or concluding that the patient will have a short disease-free survival when the level determined at step i) is equal or higher than the predetermined reference value.

As used herein, the “reference value” refers to a threshold value or a cut-off value. The setting of a single “reference value” thus allows discrimination between a poor and a good prognosis with respect to the overall survival (OS) for a patient. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the expression level (obtained according to the method of the invention) with a defined threshold value. In one embodiment of the present invention, the threshold value is derived from the expression level (or ratio, or score) determined in a biological sample derived from one or more patients having pancreatic ductal adenocarcinoma (PDA). Furthermore, retrospective measurement of the expression level (or ratio, or scores) in properly banked historical patient samples may be used in establishing these threshold values.

Predetermined reference values used for comparison may comprise “cut-off” or “threshold” values that may be determined as described herein. Each reference (“cut-off”) value for the biomarker of interest may be predetermined by carrying out a method comprising the steps of
a) providing a collection of samples from patients suffering of pancreatic ductal adenocarcinoma (PDA);
b) determining the expression level of REG3A for each sample contained in the collection provided at step a);
c) ranking the tumor tissue samples according to said expression level;
d) classifying said samples in pairs of subsets of increasing, respectively decreasing, number of members ranked according to their expression level,
e) providing, for each sample provided at step a), information relating to the responsiveness of the patient or the actual clinical outcome for the corresponding cancer patient (i.e. the duration of the event-free survival (EFS), metastasis-free survival (MFS) or the overall survival (OS) or both);
f) for each pair of subsets of samples, obtaining a Kaplan Meier percentage of survival curve;
g) for each pair of subsets of samples calculating the statistical significance (p value) between both subsets;
h) selecting as reference value for the expression level, the value of expression level for which the p value is the smallest.

For example the expression level of a biomarker has been assessed for 100 PDA samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding PDA patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate PDA samples and therefore the corresponding patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the expression level of a biomarker should of course be used for obtaining the reference value and thereafter for assessment of the expression level of a biomarker of a patient subjected to the method of the invention.

In some embodiment, the reference value is 17.5 μg/mL.

In some embodiment, the present invention relates to a method for predicting the survival time of a patient suffering from pancreatic ductal adenocarcinoma (PDA) comprising the steps of: i) determining the expression level of REG3A in a biological sample obtained from the patient, ii) comparing the expression level determined at step i) with a predetermined reference value and iii) concluding that the patient will have a long survival time when the level determined at step i) is lower than 17.5 μg/mL or concluding that the patient will have a short survival time when the level determined at step i) is equal or higher than 17.5 μg/mL.

In some embodiments, the present invention relates to a method for predicting the disease-free survival (DFS) of a PDA resected patient comprising the steps of: i) determining the expression level of REG3A in a biological sample obtained from the patient, ii) comparing the expression level determined at step i) with a predetermined reference value and iii) concluding that the patient will have a long disease-free survival when the level determined at step i) is lower than 17.5 μg/mL or concluding that the patient will have a short disease-free survival when the level determined at step i) is equal or higher than 17.5 μg/mL.

Analyzing the REG3A expression level may be assessed by any of a wide variety of well-known methods for detecting expression of a transcribed nucleic acid or translated protein.

In one embodiment, the REG3A expression level is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarker.

Methods for measuring the expression level of a biomarker in a sample may be assessed by any of a wide variety of well-known methods from one of skill in the art for detecting expression of a protein including, but not limited to, direct methods like mass spectrometry-based quantification methods, protein microarray methods, enzyme immunoassay (EIA), radioimmunoassay (RIA), Immunohistochemistry (IHC), Western blot analysis, ELISA, Luminex, ELISPOT and enzyme linked immunoabsorbant assay and undirect methods based on detecting expression of corresponding messenger ribonucleic acids (mRNAs). The mRNA expression profile may be determined by any technology known by a man skilled in the art. In particular, each mRNA expression level may be measured using any technology known by a man skilled in the art, including nucleic microarrays, quantitative Polymerase Chain Reaction (qPCR), next generation sequencing and hybridization with a labelled probe.

Said direct analysis can be assessed by contacting the sample with a binding partner capable of selectively interacting with the biomarker present in the sample. The binding partner may be an antibody that may be polyclonal or monoclonal, preferably monoclonal (e.g., a isotope-label, element-label, radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarker of the invention. In another embodiment, the binding partner may be an aptamer.

The binding partners of the invention such as antibodies or aptamers, may be labelled with a detectable molecule or substance, such as an isotope, an element, a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as an isotope, an element, a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be produced with a specific isotope or a radioactive molecule by any method known in the art. For example radioactive molecules include but are not limited to radioactive atom for scintigraphic studies such as 1123, 1124, In111, Re186, Re188, specific isotopes include but are not limited to 13C, 15N, 126I, 79Br, 81Br.

The afore mentioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidene fluoride; diazotized paper; nylon membranes; activated beads, magnetically responsive beads, silicon wafers.

In a particular embodiment, an ELISA method can be used, wherein the wells of a microtiter plate are coated with a set of antibodies which recognize said biomarker. A sample containing or suspected of containing said biomarker is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art such as Singulex, Quanterix, MSD, Bioscale, Cytof.

In one embodiment, an Enzyme-linked immunospot (ELISpot) method may be used. Typically, the sample is transferred to a plate which has been coated with the desired anti-biomarker capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-biomarker expression measurement is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, Calif.). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In one embodiment, protein microarray methods may be used. Typically, at least one antibody or aptamer directed against the biomarker is immobilized or grafted to an array(s), a solid or semi-solid surface(s). A sample containing or suspected of containing the biomarker is then labelled with at least one isotope or one element or one fluorophore or one colorimetric tag that are not naturally contained in the tested sample. After a period of incubation of said sample with the array sufficient to allow the formation of antibody-antigen complexes, the array is then washed and dried. After all, quantifying said biomarker may be achieved using any appropriate microarray scanner like fluorescence scanner, colorimetric scanner, SIMS (secondary ions mass spectrometry) scanner, maldi scanner, electromagnetic scanner or any technique allowing to quantify said labels.

In another embodiment, the antibody or aptamer grafted on the array is labelled.

In another embodiment, reverse phase arrays may be used. Typically, at least one sample is immobilized or grafted to an array(s), a solid or semi-solid surface(s). An antibody or aptamer against the suspected biomarker is then labelled with at least one isotope or one element or one fluorophore or one colorimetric tag that are not naturally contained in the tested sample. After a period of incubation of said antibody or aptamer with the array sufficient to allow the formation of antibody-antigen complexes, the array is then washed and dried. After all, detecting quantifying and counting by D-SIMS said biomarker containing said isotope or group of isotopes, and a reference natural element, and then calculating the isotopic ratio between the biomarker and the reference natural element. may be achieve using any appropriate microarray scanner like fluorescence scanner, colorimetric scanner, SIMS (secondary ions mass spectrometry) scanner, maldi scanner, electromagnetic scanner or any technique allowing to quantify said labels.

In one embodiment, said direct analysis can also be assessed by mass Spectrometry. Mass spectrometry-based quantification methods may be performed using either labelled or unlabelled approaches (DeSouza and Siu, 2012). Mass spectrometry-based quantification methods may be performed using chemical labeling, metabolic labelingor proteolytic labeling. Mass spectrometry-based quantification methods may be performed using mass spectrometry label free quantification, LTQ Orbitrap Velos, LTQ-MS/MS, a quantification based on extracted ion chromatogram EIC (progenesis LC-MS, Liquid chromatography-mass spectrometry) and then profile alignment to determine differential expression of the biomarker.

In another embodiment, the biomarker expression level is assessed by analyzing the expression of mRNA transcript or mRNA precursors, such as nascent RNA, of biomarker gene. Said analysis can be assessed by preparing mRNA/cDNA from cells in a sample from a patient, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the gene encoding for biomarkers involves the process of nucleic acid amplification, e. g., by RT-PCR (the experimental embodiment set forth in U.S. Pat. No. 4,683,202), ligase chain reaction (Barany, 1991), self sustained sequence replication (Guatelli et al., 1990), transcriptional amplification system (Kwoh et al., 1989), Q-Beta Replicase (Lizardi et al., 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In a further aspect, the present invention relates to a method of determining whether the pancreatic ductal adenocarcinoma (PDA) of a patient is a low risk tumor or a high risk tumor, comprising the steps of: (i) determining the expression level of REG3A in a biological sample obtained from the patient, (ii) comparing the expression level of REG3A in the biological sample with a predetermined reference value, and (iii) concluding that the pancreatic ductal adenocarcinoma (PDA) of the patient is a low risk tumor when the level determined at step i) is lower than the predetermined reference value or concluding that pancreatic ductal adenocarcinoma (PDA) of the patient is a high risk tumor when the level determined at step i) is equal or higher than the predetermined reference value.

The term “low risk PDA” or “low risk tumor” has its general meaning in the art and refers to pancreatic ductal adenocarcinoma (PDA) or tumor with low risk of clinically aggressive behavior. The term “low risk PDA” also refers to PDA in a patient with high overall survival, progression-free survival (PFS) and/or disease-free survival (DFS). The term “low risk PDA” refers to PDA in a patient with low relapse risk. The term “low risk PDA” also refers to PDA in a patient with high relapse-free overall survival. The term “low risk PDA” also refers to PDA in a patient with low cancer cell migration and invasion abilities, low cancer cell aggressiveness, and/or low peri-neural invasion.

The term “high risk PDA” or “high risk tumor” has its general meaning in the art and refers to pancreatic ductal adenocarcinoma (PDA) or tumor with high risk of clinically aggressive behavior. The term “high risk PDA” also refers to PDA in a patient with reduced overall survival, progression-free survival (PFS) and/or disease-free survival (DFS). The term “high risk PDA” refers to PDA in a patient with high relapse risk. The term “high risk PDA” also refers to PDA in a patient with reduced relapse-free overall survival. The term “high risk PDA” also refers to PDA in a patient with high cancer cell migration and invasion abilities, high cancer cell aggressiveness, and/or high peri-neural invasion.

A further aspect of the invention relates to a method of monitoring pancreatic ductal adenocarcinoma (PDA) progression by performing the method of the invention.

A further aspect of the invention relates to a method of determining whether a patient afflicted with pancreatic ductal adenocarcinoma (PDA) will be a responder or a non-responder to JAK2/STAT3 signaling inhibitor treatment comprising the step of measuring the expression level of REG3A in a biological sample obtained from said patient.

In some embodiments, the method of the invention is performed before the JAK2/STAT3 signaling inhibitor treatment.

In some embodiments, the method of the invention is performed during the JAK2/STAT3 signaling inhibitor treatment.

The term “responder” refers to a patient afflicted with pancreatic ductal adenocarcinoma (PDA) that will respond to JAK2/STAT3 signaling inhibitor treatment. The disease activity can be measured according to the standards recognized in the art. The disease activity may be measured by clinical and physical examination, biochemical analyses (ACE, Ca 19-9, albuminemia, bilirubinemia), blood analysis, immunostaining, immunoblots, progression-free survival, overall survival and characteristics of the patient and tumor as described in the example. A “responder” or “responsive” patient to a JAK2/STAT3 signaling inhibitor treatment refers to a patient who shows or will show a clinically significant relief in the disease when treated with JAK2/STAT3 signaling inhibitor. The term “responder” also refers to a patient having longer stable disease or higher relapse-free overall survival after JAK2/STAT3 signaling inhibitor treatment. The term “responder” also refers to a patient having longer overall survival, progression-free survival (PFS) and/or disease-free survival (DFS) after JAK2/STAT3 signaling inhibitor treatment. The term “responder” also refers to a patient with low cancer cell migration and invasion abilities, low cancer cell aggressiveness, and/or low peri-neural invasion.

The method of the invention may further comprise a step consisting of comparing the expression level of REG3A in the biological sample with a reference value, wherein detecting differential in the expression level of the REG3A between the biological sample and the reference value is indicative that said subject will be a responder or a non-responder.

In one embodiment, higher expression level of REG3A is indicative that the subject will be a responder to JAK2/STAT3 signaling inhibitor treatment, and accordingly lower expression level of REG3A is indicative that the subject will be a non-responder to JAK2/STAT3 signaling inhibitor treatment.

In a further aspect, the present invention relates to a REG3A inhibitor for use in the treatment of high risk pancreatic ductal adenocarcinoma (PDA) in a patient in need thereof wherein the patient was being classified as having a high risk tumor by the method as above described.

As used herein, the term “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The term “REG3A inhibitor” has its general meaning in the art and refers to a compound that selectively blocks or inactivates the REG3A. The term “REG3A inhibitor” also refers to a compound that selectively blocks the binding of REG3A to its receptors (such as gp130). The term “REG3A inhibitor” also refers to a compound able to prevent the action of REG3A for example by inhibiting the REG3A controls of downstream effectors such as inhibiting the activation of the IL-6 and JAK2/STAT3 signaling. As used herein, the term “selectively blocks or inactivates” refers to a compound that preferentially binds to and blocks or inactivates REG3A with a greater affinity and potency, respectively, than its interaction with the other sub-types of the REG family. Compounds that block or inactivate REG3A, but that may also block or inactivate other REG3A sub-types, as partial or full inhibitors, are contemplated. The term “REG3A inhibitor” also refers to a compound that inhibits REG3A expression. Typically, a REG3A inhibitor is a small organic molecule, a polypeptide, an aptamer, an antibody, an oligonucleotide or a ribozyme.

Tests and assays for determining whether a compound is a REG3A inhibitor are well known by the skilled person in the art such as described in Liu et al., 2015; Wang et al., 2014; Ye et al., 2015.

In one embodiment of the invention, REG3A inhibitors include but are not limited to the anti-Reg3a antibodies ab95316 and Ab134309 (Abcam, Cambridge, Mass., USA) and antibodies such as described in Liu et al., 2015; Wang et al., 2014; and Ye et al., 2015.

In some embodiments, the REG3A inhibitor is a gp130 antagonist or a JAK2/STAT3 signaling inhibitor.

The term “gp130” has its general meaning in the art and refers to CD130, the cytokine leukemia inhibitory factor (LIF) subunit complex receptor (Nicolas and Babon, 2015).

The term “gp130 antagonist” has its general meaning in the art and refers to compounds such as quinoxalinhydrazide derivative SC144 having the general formula (I), AG490 having the general formula (II), soluble forms of gp130 (sgp130) and compounds described in Seo et al., 2009; Xu et al., 2013; Huang et al., 2010; Femandez-Botran, 2000; Xu and Neamati, 2013.

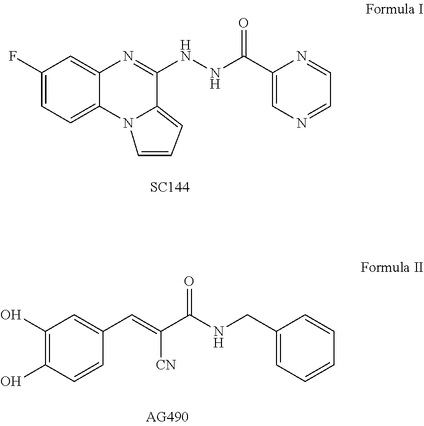

The term “JAK2/STAT3 signaling inhibitor” has its general meaning in the art and refers to compounds such as JAK2 inhibitors and STAT3 antagonists.

JAK2 inhibitors are well known in the art (Tibes R, Bogenberger J M, Geyer H L, Mesa R A. JAK2 inhibitors in the treatment of myeloproliferative neoplasms. Expert Opin Investig Drugs. 2012 December; 21(12):1755-74; Dymock B W, See CS. Inhibitors of JAK2 and JAK3: an update on the patent literature 2010-2012. Expert Opin Ther Pat. 2013 April; 23(4):449-501) and include but are not limited to ruxolitinib (INCB018424), SAR302503 (TG101348), Pacritinib (SB1518), CYT387, AZD-1480, BMS-911543, BMS-91153, NS-018, LY2784544, Lestaurtinib (CEP701), AT-9283, CP-690550, SB1578, R723, INCB16562, INCB20, CMP6, TG101209, SB1317 (TG02), XL-019, Baricitinib (LY3009104, INCB28050), AG490 and compounds described in WO2012030944, WO2012030924, WO2012030914, WO2012030912, WO2012030910, WO2010099379, WO2012030894, WO2010002472, WO2011130146, WO2010038060, WO2010020810, WO2011028864, WO2010141796, WO2010071885, WO2011101806, S20100152181, WO2010010190, WO2010010189, WO2010051549, WO2011003065, WO2012022265, WO2012068440, WO2011028685, WO2010135621, WO2010039939, WO2012068450, WO2011103423, WO2011044481, WO2010085597, WO2010014453, WO2010011375, WO2010069966, WO2011097087, WO2011075334, WO2011045702, WO2010020905, WO2010039518, WO2010068710.

STAT3 antagonists are well-known in the art as illustrated by Yu W., J Med Chem. 2013 May 7; Turkson et al., Mol Cancer Ther. 2004 March; 3(3):261-9; McMurray J S. Chem Biol. 2006 November; 13(11):1123-4; Liu A, Cancer Sci. 2011 July; 102(7):1381-7; Song H., Proc Natl Acad Sci USA. 2005 Mar. 29; 102(13); and Wang X., Int J Oncol. 2012 July; 24.

The term “STAT3 antagonists” refers to compounds such as compounds that inhibit STAT3 phosphorylation such as PM-73G and pCinn-Leu-cis-3,4-methanoPro-Gln-NHBn (Yu W., J Med Chem. 2013 May 7); and non-peptidomimetic small inhibitors such as 5-hydroxy-9,10-dioxo-9,10-dihydroanthracene-1-sulfonamide (LLL12) and a steroidal natural product such as cucurbitacin (McMurray J S. Chem Biol. 2006 November; 13(11):1123-4; Yu W., J Med Chem. 2013 May 7).

The term “STAT3 antagonists” also refers to compounds that inhibit STAT3 dimerization such as peptidomimetics XZH-5 (Yu W., J Med Chem. 2013 May 7); ISS 610; ISS 219 and compounds described in Turkson et al., Mol Cancer Ther. 2004 March; 3(3):261-9; and small molecules such as Stattic; STA-2; LLL-3; S31-201 (NSC 74859); S31-20; S31-201.1066; S3I-M200; 5,15-DPP; STX-0119; Niclosamide (Siddiquee K A., ACS Chem Biol. 2007 Dec. 21; 2(12):787-98; Yu W., J Med Chem. 2013 May 7).

The term “STAT3 antagonists” refers to compounds such as 5,8-dioxo-6-(pyridin-3-ylamino)-5,8-dihydronaphthalene-1-sulfonamide (LY5); Naphthalene-5,8-dione-1-sulfonamide (Naphthalenesulfonylchloride); 5,8-dioxo-6-(phenylamino)-5,8-dihydronaphthalene-1-sulfonamide; 5H-Naphth[1,8-cd]isothiazol-5-one, 1,1-dioxide, 6-(phenylamino); 5H-Naphth[1,8-cd]isothiazol-5-one, 1,1-dioxide, 6-(1′-chloro-3′-nitro-2′-phenylamino); 5H-Naphth[1,8-cd]isothiazol-5-one, 1,1-dioxide, 6-(naphthylamino); Niclosamide (Yu W., J Med Chem. 2013 May 7); FLLL31; FLLL32 (Liu A, Cancer Sci. 2011 July; 102(7):1381-7); NCT00511082; NCT00657176; NCT00955812; NCT01029509; NCT00696176 (Wang X., Int J Oncol. 2012 Jul. 24).

In another embodiment, the REG3A inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against REG3A of the invention as above described, the skilled man in the art can easily select those blocking or inactivating REG3A.

In another embodiment, the REG3A inhibitor of the invention is an antibody (the term including “antibody portion”) directed against REG3A.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of REG3A. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in REG3A. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a preferred embodiment, the REG3A inhibitor of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the REG3A inhibitor of the invention is a REG3A expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins (e.g., REG3A) modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene.

REG3A expression inhibitors for use in the present invention may be based on antisense oligonucleotide constructs. Anti-sense oligonucleotides, including anti-sense RNA molecules and anti-sense DNA molecules, would act to directly block the translation of REG3A mRNA by binding thereto and thus preventing protein translation or increasing mRNA degradation, thus decreasing the level of REG3A proteins, and thus activity, in a cell. For example, antisense oligonucleotides of at least about 15 bases and complementary to unique regions of the mRNA transcript sequence encoding REG3A can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as REG3A expression inhibitors for use in the present invention. REG3A gene expression can be reduced by contacting the subject or cell with a small double stranded RNA (dsRNA), or a vector or construct causing the production of a small double stranded RNA, such that REG3A expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as REG3A expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of REG3A mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful a REG3A inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing REG3A. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In one embodiment of the invention, REG3A expression inhibitors include but are not limited to siRNAs and shRNA such as described in Liu et al., 2015 and Ye et al., 2015.

Typically the inhibitors according to the invention as described above are administered to the patient in a therapeutically effective amount.

By a “therapeutically effective amount” of the inhibitor of the present invention as above described is meant a sufficient amount of the inhibitor for treating PDA at a reasonable benefit/risk ratio applicable to any medical treatment. It will be understood, however, that the total daily usage of the inhibitors and compositions of the present invention will be decided by the attending physician within the scope of sound medical judgment. The specific therapeutically effective dose level for any particular patient will depend upon a variety of factors including the disorder being treated and the severity of the disorder; activity of the specific inhibitor employed; the specific composition employed, the age, body weight, general health, sex and diet of the patient; the time of administration, route of administration, and rate of excretion of the specific inhibitor employed; the duration of the treatment; drugs used in combination or coincidential with the specific inhibitor employed; and like factors well known in the medical arts. For example, it is well within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the inhibitor of the present invention for the symptomatic adjustment of the dosage to the patient to be treated. A medicament typically contains from about 0.01 mg to about 500 mg of the inhibitor of the present invention, preferably from 1 mg to about 100 mg of the inhibitor of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the REG3A inhibitor of the present invention is administered to the patient in combination with anti-PDA treatment. The term “PDA treatment” has its general meaning in the art and refers to any type of pancreatic cancer therapy undergone by the pancreatic cancer subjects including surgical resection of pancreatic cancer, and any type of agent conventional for the treatment of PDA.

In some embodiments, the REG3A inhibitor of the present invention is administered to the patient in combination with at least one compound selected from the group consisting of gemcitabine, fluorouracil, FOLFIRINOX (fluorouracil, irinotecan, oxaliplatin, and leucovorin), nab-paclitaxel, inhibitors of programmed death 1 (PD-1), PD-1 ligand PD-L1, anti-CLA4 antibodies, EGFR inhibitors such as erlotinib, chemoradiotherapy, inhibitors of PARP, inhibitors of Sonic Hedgehog, gene therapy and radiotherapy.

In a further aspect, the present invention relates to a method of screening a candidate compound for use as a drug for treating PDA in a patient in need thereof, wherein the method comprises the steps of:
providing a REG3A, providing a cell, tissue sample or organism expressing a REG3A,
providing a candidate compound such as a small organic molecule, a polypeptide, an aptamer, an antibody or an intra-antibody,
measuring the REG3A activity,
and selecting positively candidate compounds that inhibit REG3A activity.

Methods for measuring REG3A activity are well known in the art (Liu et al., 2015; Wang et al., 2014; Ye et al., 2015). For example, measuring the REG3A activity involves determining a Ki on the REG3A cloned and transfected in a stable manner into a CHO cell line, measuring cancer cell migration and invasion abilities, measuring peri-neural invasion, and measuring JAK2/STAT3 signaling in the present or absence of the candidate compound.

Tests and assays for screening and determining whether a candidate compound is a REG3A inhibitor are well known in the art (Liu et al., 2015; Wang et al., 2014; Ye et al., 2015). In vitro and in vivo assays may be used to assess the potency and selectivity of the candidate compounds to inhibit REG3A activity.

Activities of the candidate compounds, their ability to bind REG3A and their ability to inhibit REG3A activity may be tested using isolated cancer cell or CHO cell line cloned and transfected in a stable manner by the human REG3A.

Activities of the candidate compounds and their ability to bind to the REG3A may be assessed by the determination of a Ki on the REG3A cloned and transfected in a stable manner into a CHO cell line, measuring cancer cell migration and invasion abilities, measuring peri-neural invasion in the present or absence of the candidate compound. The ability of the candidate compounds to inhibit REG3A activity may be assessed by measuring JAK2/STAT3 signaling such as described in the example.

Cells expressing another cytokine than REG may be used to assess selectivity of the candidate compounds.

The inhibitors of the invention may be used or prepared in a pharmaceutical composition.

In one embodiment, the invention relates to a pharmaceutical composition comprising the inhibitor of the invention and a pharmaceutical acceptable carrier for use in the treatment of high risk pancreatic ductal adenocarcinoma (PDA) in a patient of need thereof.

Typically, the inhibitor of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, intramuscular, intravenous, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, intraperitoneal, intramuscular, intravenous and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active inhibitors in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular and intraperitoneal administration. In this connection, sterile aqueous media which can be employed will be known to those of skill in the art in light of the present disclosure. Some variation in dosage will necessarily occur depending on the condition of the patient being treated. The person responsible for administration will, in any event, determine the appropriate dose for the individual patient.

In addition to the inhibitors of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further compound which is used in the treatment of pancreatic ductal adenocarcinoma.

In one embodiment, said additional active compounds may be contained in the same composition or administrated separately.

In another embodiment, the pharmaceutical composition of the invention relates to combined preparation for simultaneous, separate or sequential use in the treatment of high risk pancreatic ductal adenocarcinoma (PDA) in a patient in need thereof.

The invention also provides kits comprising the inhibitor of the invention. Kits containing the inhibitor of the invention find use in therapeutic methods.

In a further aspect, the present invention relates to a REG3A inhibitor for use in the prevention of progression of low risk pancreatic ductal adenocarcinoma (PDA) to high risk pancreatic ductal adenocarcinoma (PDA) in a patient in need thereof wherein the patient was being classified as having a high risk tumor by the method as above described.

In one embodiment, the present invention relates to a method of treating high risk pancreatic ductal adenocarcinoma (PDA) in a patient in need thereof comprising the steps of:

i) determining whether the pancreatic ductal adenocarcinoma (PDA) of a patient is a high risk tumor by performing the method according to the invention, and

ii) administering a REG3A inhibitor if said patient was being classified as having a high risk tumor.

In one embodiment, the present invention relates to a method of preventing the progression of low risk pancreatic ductal adenocarcinoma (PDA) to high risk pancreatic ductal adenocarcinoma (PDA) in a patient in need thereof comprising the steps of:

i) determining whether the pancreatic ductal adenocarcinoma (PDA) of a patient is a low risk tumor or a high risk tumor by performing the method according to the invention, and

ii) administering a REG3A inhibitor if said patient was being classified as having a high risk tumor.

The method of the invention allows to define a subgroup of patients who will be responder or non responder to JAK2/STAT3 signaling inhibitor treatment.

A further aspect of the invention relates to a method for treating pancreatic ductal adenocarcinoma (PDA) in a patient in need thereof comprising the steps of:

a) determining whether a patient afflicted with pancreatic ductal adenocarcinoma (PDA) will be a responder or a non-responder to JAK2/STAT3 signaling inhibitor treatment by performing the method according to the invention,

b) administering the JAK2/STAT3 signaling inhibitor treatment, if said patient has been considered as a responder.

The invention will be further illustrated by the following figures and examples. However, these examples and figures should not be interpreted in any way as limiting the scope of the present invention.

### FIGURES

FIG. 1: High PAP/REG3A levels are associated with shorten survival and tumor grade in PDA. A. Quantification of PAP/REG3A (μg/L) level in blood obtained from healthy donors (n=85) and patients with PDA from 3 different cohorts (1, n=74; 2, n=34; 3, n=54) (median+interquartile range). B. Kaplan-Meier overall survival curve using PAP/REG3A measured in serum from patients with PDA from cohort 1, divided into high (≥17.5 μg/L) and low (<17.5 μg/L) groups (n=27 and 52, respectively). C. Kaplan-Meier overall survival curve using PAP/REG3A measured in serum from patients with PDA from cohort 2, divided into high (≥17.5 μg/L) and low (<17.5 μg/L) groups (n=18 and 15, respectively). D. Quantification of PAP/REG3A measured in serum from patients with stage 1, 2 and 3 PDA from cohort 1, divided into high (≥17.5 μg/L) and low (<17.5 μg/L) PAP/REG3A groups (n=27 and 52, respectively). E. Quantification of resectable and non-resectable PDA in high (≥17.5 μg/L) and low (<17.5 g g/L) PAP/REG3A groups from cohort 1 (n=27 and 52, respectively).

FIG. 2: PAP/REG3A enhances PDA cancer cell aggressiveness. A. Mouse pancreatic cancer cell, Pk4A, migration ability in presence of various mouse PAP/REG33 recombinant protein concentration with a range from 20 ng/mL to 500 ng/mL (median+interquartile range, n=3). *, P<0.05; **, P<0.01. B. Human pancreatic cancer cell, Panc-1, migration ability in presence of various Human PAP/REG3A recombinant protein concentration with a range from 20 ng/mL to 500 ng/mL (median+interquartile range, n=3). **, P<0.01; ***, P<0.001. C. Human pancreatic cancer cell, Panc-1, migration ability+100 ng/mL of mouse PAP/REG3A recombinant protein+AG490 treatment (median+interquartile range, n=3). *, P<0.05; **, P<0.01. D. Mouse pancreatic cancer cell, Pk4A, invasion ability+500 ng/mL of human PAP/REG3β recombinant protein+AG490 treatment (median+interquartile range, n=3). *, P<0.05; **, P<0.01. E. Mouse pancreatic cancer cell, Pk4A, migration ability+control or PAP/REG3β-depleted acinar cell media (ACm/PAP+ or ACm/PAP−, respectively)+AG490 treatment (median+interquartile range, n=3). *, P<0.05.

FIG. 3: PAP/REG3A increases peri-neural invasion (PNI) and is associated with worst prognosis for resected patients. A. Measurement of peri-neural invasion ability of human pancreatic cancer cell, Panc-1, using an ex-vivo PNI assay in presence or not of human PAP/REG3A recombinant protein (500 ng/mL) and AG490 treatment (median+interquartile range, n=3). *, P<0.05. B. Measurement of peri-neural invasion ability of human pancreatic cancer cell, Panc-1, using an ex-vivo PNI assay in presence of control or PAP/REG3A-depleted acinar cell media (ACm/PAP+ or ACm/PAP−, respectively) and SC144 treatment (median+interquartile range, n=3). *, P<0.05. C. Kaplan-Meier disease free survival curve using PAP/REG3A measured in serum from patients with resected PDA from cohort 1, 2 and 3, divided into high (≥17.5 μg/L) and low (<17.5 μg/L) groups (n=1 and 24, respectively). D. Kaplan-Meier overall survival curve following resection using PAP/REG3A measured in serum from patients with resected PDA from cohort 2, divided into high (≥17.5 μg/L) and low (<17.5 μg/L) groups (n=8 and 6, respectively). E. Kaplan-Meier overall survival curve following resection using PAP/REG3A measured in serum from patients with resected PDA from cohort 3, divided into high (≥17.5 μg/L) and low (<17.5 μg/L) groups (n=10 and 12, respectively).

### EXAMPLE

Material & Methods

Mouse Strains and Tissue Collection

Pdx1-Cre;Ink4a/Arf^{fl/fl};LSL-Kras^{G12D} mice were obtained by crossing the following strains: Pdx1-Cre;Ink4a/Arf^{fl/fl} and LSL-Kras^{G12D} mice kindly provided by Dr. D. Melton (Harvard Stem Cell Institute, Cambridge, Mass.), Dr. R. Depinho (Dana-Farber Cancer Institute, Boston) and Dr. T Jacks (David H. Koch Institute for Integrative Cancer Research, Cambridge, Mass.), respectively. PDAC-bearing 8-12 week-old male mice were killed with their mating control littermates. Pieces of tumor or control pancreas were fixed in 4% formaldehyde for further immunostaining analysis. For isolation of acinar cells, control mice were killed between 8 and 12 week-old. All animal care and experimental procedures were performed in agreement with the Animal Ethics Committee of Marseille number 13.

Human Tissue Samples

Human pancreatic adenocarcinoma, used for immunostaining or immunoblots, were collected at surgery from 36 PDA patients with available clinical history, at the Gastroenterology Department of the Hôpital Nord de Marseille, France. All tissues were collected via standardized operative procedures approved by the Institutional Ethical Board and in accordance with the Declaration of Helsinki. Informed consent was obtained for all tissue samples linked with clinical data.

Blood Sample Cohorts

For cohort 1, from January 2011 to April 2015, a translational research study of blood samples was proposed to all consecutive patients (n=79) with a histologically proved pancreatic adenocarcinoma and treated in “Hôpital Pitié-Salpétrière” (Paris, France). This translational research was approved by the local ethic committee (“Comité de Protection des Personnes Ile de France IV”). After acceptance and signature of informed consent, blood was collected directly from the vena cava and the central catheter on the day of the first chemotherapy cycle. For the patients with pancreatic adenocarcinoma who underwent curative surgical resection, blood samples were collected after the surgery, the day of the first cycle of adjuvant chemotherapy. For the patients with an advanced pancreatic adenocarcinoma (locally advanced or metastatic), blood samples were collected the day of the first cycle of chemotherapy. Two EDTA tubes and two BD® P100 tubes were used. After the blood sampling, as soon as possible and always within 3 hours from collection, the EDTA and BD® P100 tubes were centrifuged at 3,500 rpm for 15 min at 4° C. Plasma were collected and stored at −80° C. in several aliquots (2-ml Eppendorf tubes) at the biological resources center (CRB) of the treatment center. Data from medical records were recorded in a database. The following information was collected prospectively: characteristics of the patients and tumor at inclusion (gender, age, medical history, date of diagnosis, location of the primary tumor, primary tumor diameter, tumor differentiation grade, stage of the disease), biologic data before first chemotherapy cycle (ACE, Ca 19-9, albuminemia, bilirubinemia) and data of follow-up (date of primary resection, date and type of relapse, date of diagnosis of metastasis, date and type of chemotherapy regimen, date and type of chemoradiotherapy, date of death or last follow-up).

For cohort 2, plasma samples of the Brussels cohort were obtained from patients with chronic pancreatitis (n=20), and patients with histologically proven PADC (n=34) before receiving any treatment. Among patients diagnosed with PADC, 14 underwent PADC resection and 20 had a metastatic disease. Plasma samples are prospectively collected in Erasme University Hospital at time of diagnosis, and stored under and according to rigorous standard operating procedures. Clinical and pathological data are prospectively collected and regularly updated. All research samples were collected after obtaining written informed consent for participation in accordance with the Declaration of Helsinki, and with ethical approval from the local institutional review boards (ref:B2011/005).

For cohort 3, plasma samples were obtained from patients diagnosed of PDAC (n=54) in Hospital Clinic of Barcelona before receiving any treatment. All specimens were obtained according to the Institutional Review Board-approved procedures for consent. Ethically approved informed consent was obtained from all subjects and all the experiments conformed to the principles set out in the WMA Declaration of Helsinki. Data from medical records were recorded in a database. Blood samples were collected in tubes containing EDTA and plasma were separated by two consecutive centrifuges (1,600×g for 10 min at 4° C. followed by further centrifugation at 16,000×g for 10 minutes at 4° C. to completely remove cellular components). Plasma was then aliquoted and stored at −80° C. until use.

Blood samples of healthy persons were collected by EFS (Etablissement Français du Sang).

PAP/REG3β Measurement from Blood Samples and Culture Medium

Human PAP/REG3β concentration was measured in blood samples using a commercially available ELISA kit PANCREPAP (Dynabio SA, Marseille, FR) following the manufacturer's instructions. Results were expressed as μg of PAP/REG33 per L of plasma (μg/L). Plasma samples were diluted 1/200. All samples were run in triplicate and a standard curve was established for each assay. The absorbance was measured on the Thermo Scientific™ Multiskan™ Spectrum spectrophotometer.

Cell Culture

Human pancreatic cancer (Panc-1) cell line was obtained from American Type Culture Collection (ATCC). PK4A cells were isolated from Pdx1-Cre;LSL-Kras^{G12D};Ink4a/Arf^{fl/fl} pancreatic ductal adenocarcinoma (PDA) as described previously [27]. Panc-1 and PK4A were cultivated in DMEM medium (Thermofisher, Waltham, Mass., USA) supplemented with 10% Fetal Bovine Serum (A15-151, GE Healthcare, Little Chalfont, GB) and 1% of antibiotic/antimycotic (Thermofisher, Waltham, Mass., USA).

Pancreatic acinar cells were isolated from control LSL-Kras^{G12D};Ink4a/Arf^{fl/fl} mice (according to Gout et al [28]), and cultivated in Waymouth's medium (Thermofisher, Waltham, Mass., USA) supplemented with 2.5% Fetal Bovine Serum (GE Healthcare, Little Chalfont, GB), 1% Penicillin-Streptomycin mixture (Ser. No. 15/140,122, Thermofisher, Waltham, Mass., USA), 0.25 mg/mL of trypsin inhibitor (T6522, Sigma-Aldrich, St Quentin, FR) and 25 ng/mL of recombinant human Epidermal Growth Factor (EGF) from Promocell (C-60180, Heidelberg, GE). Acinar cell conditioned media were realized over 24 hours in similar media as depicted above but with only 1% FBS. For PAP/REG3A depletion, conditioned media were incubated 2 h with 10 μg/mL of a Rabbit anti-PAP/REG3A antibody (gift from Dynabio SA, Marseille, FR) at 4° C. then 30 min with 17 μl of Protein G-Agarose from Thermofisher (20398, Waltham, Mass., USA). Conditioned media were centrifuged 3 min at 600 g. Supernatants were recovered and called ACm/PAP−.

Reagents

Mouse PAP/REG3β recombinant protein was purchased from R&D systems (5110-RG-050, Lille, FR) while human PAP/REG3A recombinant proteins was a kind gift from Dynabio SA (Marseille, FR). Tyrphostin (AG490), a JAK2/STAT3 inhibitor, was purchased from Sigma-Aldrich (T3434-5MG, St Quentin, FR) and used at 30 μM while sc144 hydrochloride, an inhibitor of glycoprotein gp130, was purchased from Sigma-Aldrich (SML0763-5MG, St Quentin, FR) and used at 2 μM.

Immunohistochemistry

5 μm formalin-fixed, paraffin-embedded human or mouse sections were deparaffinized in xylene and rehydrated through a graded ethanol series. An antigen retrieval step from Dako (S1699, Glostrup, DK) for PAP/REG3A and β staining or from Diapath (T0050, Bergamo, IT) for gp130 and pgp9.5 staining was performed before quenching endogenous peroxidase activity [3% (vol/vol) H2O2]. Tissue sections were then incubated with primary antibody, and immunoreactivities were visualized using the Vectastain ABC kit from Vector Laboratories (PK-4001, Burlingame, Calif., USA) or Streptavidin-HRP from Dako (P0397, Glostrup, DK) according to the manufacturers' protocol. Peroxidase activity was revealed using the liquid diaminobenzidine substrate chromogen system from Dako (K3468, Glostrup, DK). Counter staining with Mayer hematoxylin from Merck (1.09249.0500, Darmstadt, GE) was followed by a bluing step in 0.1% sodium bicarbonate buffer, before final deshydration, clearance, and mounting of the sections with Pertex500 from Histolab (Goteborg, SE). Dilutions of primary antibodies: Rabbit anti-PAP/REG3A and β (1/150, Dynabio SA, Marseille, FR), mouse anti-gp130 from Santa Cruz Biotechnology (sc-376280, 1/50, Dallas, Tex., USA) and rabbit anti-PGP9.5 from Abcam (ab10404, 1/800, Cambridge, GB).

Immunofluorescence

5 μm formalin-fixed, paraffin-embedded human or mouse sections were deparaffinized in xylene and rehydrated through a graded ethanol series. An antigen retrieval step with 10 mM sodium citrate from Diapath (T0050, Bergamo, IT) and 0.05% Tween 20 from Euromedex (2001-B, Souffelweyersheim, FR) at 95° C. was then performed before tissue sections were pre-incubated in blocking solution [3% (wt/vol) BSA from Sigma-Aldrich (A7284, St Quentin, FR)/10% (vol/vol) goat serum from Abcam (ab7481, Cambridge, GB)] for 1 h. Tissue sections were incubated in a mixture of two primary antibodies; one against PAP/REG3A/3 and one against either alpha smooth muscle actin (αSMA, 1:200) from Sigma-Aldrich (A2547, St Quentin, FR), alpha amylase (αAMYL, 1/400) from Abcam (ab21156, Cambridge, GB), cytokeratin (PanCK, 1/50) from Dako (M3515, Glostrup, DK), CD68 (1/50) from Abcam (ab955, Cambridge, GB), NF200 (1/200) from Sigma-Aldrich (N0142, St Quentin, FR) or neurofilament (NF, 1/50) from Clinisciences (Mob080, Nanterre, FR) in blocking solution overnight at 4° C. After washing in PBS, slides were incubated with a mixture of two secondary antibodies in blocking solution (Alexa Fluor 568-conjugated or Alexa Fluor 488-conjugated antibody, 1/500, Molecular Probes, Waltham, Mass., USA). Stained tissue sections were mounted using Prolong Gold Antifade reagent with DAPI (Life Technologies) before being sequentially scanned at a 20× magnification under a fluorescent microscope (Nikon Eclipse 90i) equipped with a CCD camera (Nikon DS-1QM).

Cell Migration Assay

Cancer cell migration was studied using PKA4 and PANC-1 cell lines under different media (medium with or without recombinant PAP/REG3A or conditioned medium from acinar cells) on Boyden chambers. Culture inserts from BD Biosciences (353097, Le pont le Claix, FR), with a porous membrane of 8 m, were coated with a mix made of gelatin 0.1% from Sigma-Aldrich (G1890, St Quentin, FR) and fibronectin 10 μg/mL from Sigma-Aldrich (F0895, St Quentin, FR) then seeded with PK4A or PANC-1 cells (100 0000 or 75 000 cells respectively per insert) and placed into wells containing culture media (with or without recombinant PAP/REG3A). PAP/REG3A or 3 were used from 0 to 500 ng/mL. Migration was performed for 4 hrs for medium with or without recombinant PAP/REG3A or β and for 1 hr 45 min for conditioned medium from acinar cells. After cleaning and briefly staining inserts with coomassie blue, migration was assessed by counting (Image J software) the number of colored cells in 8-16 high-power fields (magnification ×10).

Cell Invasion Assay

Cancer cell invasion was studied using PKA4 cells on Boyden chambers according to manufacturer's protocol (354480, Corning Lifesciences, Corning, N.Y., USA). Mouse recombinant PAP/REG3β was used at 500 ng/mL and AG490 at 30 μmol/L. Culture inserts were pre-coated with matrigel matrix then seeded with PK4A (100 0000 cells per insert) and placed into wells containing the medium. Cell counting was measured after 24 hrs of incubation. After cleaning and briefly staining inserts with coomassie blue, invasion was assessed by counting (Image J software) the number of colored cells in 8-16 high-power fields (magnification ×10).

Ex Vivo Peri-Neural Invasion Assay

The day before the experiment 125,000 PK4A cells are seeded in 24 well plates in DMEM supplemented with 10% FBS and 1% antibiotic/antimycotic. 24 hrs after inhibitors (AG490 at 30 μmol/L or SC144 at 2 μmol/L) were added for 2 h. Then culture medium is replaced by DMEM supplemented with 2% FBS, 1% antibiotic/antimycotic with or without PAP/REG3(3 (500 ng/mL) and inhibitors. A mouse sciatic nerve section (5 mm) is placed in every well and cultured for 48 hrs then nerve sections are fixed 24 hrs in 4% formaldehyde and embedded in paraffin for immunohistochemistry study. Nerve sections from each condition are cut to make 4 μm sections from 2 different depths spaced by 50 μm and fixed on slides. Slides are processed for cytokeratin immunostaining by IHC as mentioned above. Cells stained with cytokeratine inside or in contact with nerve are recorded (Image J software).

Statistical Analysis

The results showed are medians, and error bars in graphs represent standard deviations (SD). The Mann-Whitney test, recommended for the comparison of two independent groups, was performed when required. Overall and disease free survival were estimated using the Kaplan-Meier method using GraphPad Prism software. Differences were considered significant if P was less than 0.05. All P values were calculated using the GraphPad Prism software. All experiments were repeated at least 3 times.

Results

PAP/REG3A Shows a Restricted Pattern of Expression in PDA

Deciphering the role of PAP/REG3A in PDA associated nervous system alterations implies first to firmly establish which cell type/compartment would be the source of PAP/REG3A secretion. As reported previously, the cellular origin of PAP/REG3A level in PDA could be the pancreatic acinar cells adjacent to the infiltrating adenocarcinoma [24], in a peri-tumoral zone histologically resembling to chronic pancreatitis. We assessed this cellular localization through PAP/REG3A staining on human PDA slides and observed a clear staining of the peri-tumoral (PT) areas while no PAP/REG3A expression was observed in healthy (H) and intra-tumoral (IT) areas (Data not shown). This result was also obtained in PDA from Pdx1-cre/Kras^{G12D}/Ink4a^{fl/fl} mice, with a similar PT restricted staining of mice PAP/REG33 (the mice homologue of human PAP/REG3A) (Data not shown). Using several specific cell markers, we deepened previous data and determined cells from PT areas of human PDA that expressed PAP/REG3A. Co-staining of PAP/REG3A with α-amylase (inflamed acinar cell marker) were observed and confirmed the acinar cellularity of PAP/REG3A while no co-staining was observed using CAF (cSMA, alpha-smooth muscle actin), tumor cell (ck19, cytokeratin 19), M2 macrophage (cd163) or nerve (neurofilament) markers (Data not shown). Similar staining were observed using PDAs from Pdx1-cre/Kras^{G12D}/Ink4a^{fl/fl} mice (Data not shown). Those data suggest that inflamed acinar cells from peri-tumoral areas are the main mediator of PAP/REG3A secretion in PDA. In order for PAP/REG3A to efficiently activate downstream signaling in recipient cells, we analyzed and showed that gp130 (a co-receptor transducing PAP/REG3A signaling) is expressed in tumor cells and nerve compartment in human PDA (Data not shown) suggesting that both cells/structure were able to respond to PAP/REG3A. Those data are supported by the interesting observation that nerve present in or around PDA are in close contact to PT areas expressing PAP/REG3A (Data not shown). Altogether, those data revealed that PAP/REG3A is expressed by peri-tumoral compartment of PDA and can induce a response in tumor cells and nerve fibers, reinforcing the possible role of PAP/REG3A in PDA associated nervous system alterations.

High PAP/REG3A levels are associated with shorten survival and tumor grade in PDA

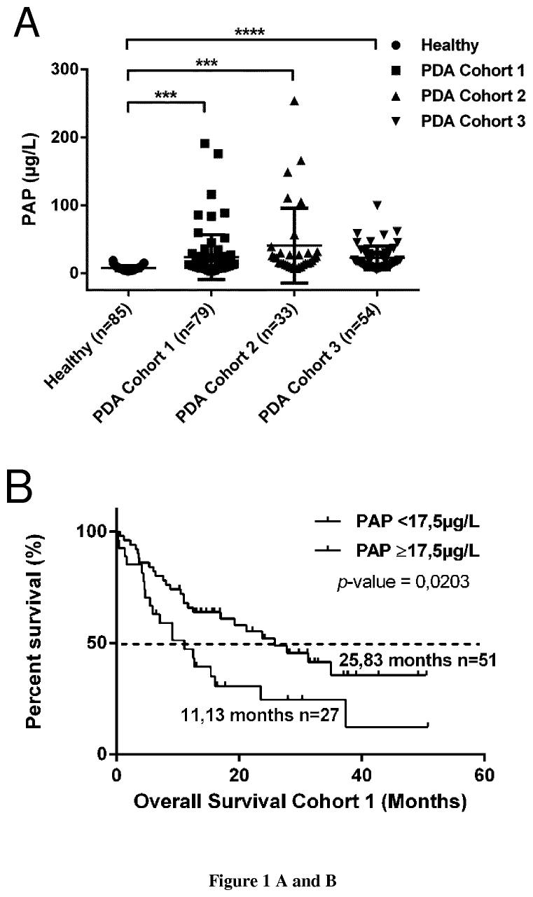

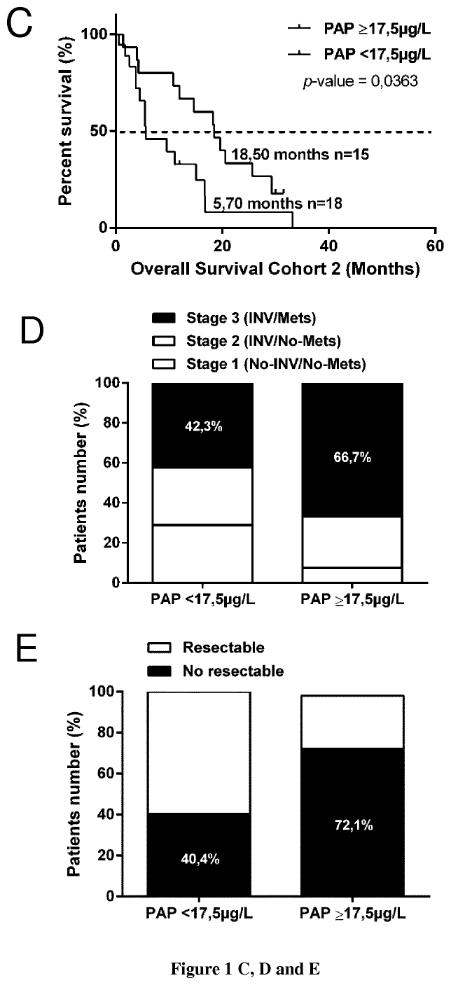

As a secreted factor, we first measured sera or plasma levels of PAP/REG3A in 85 healthy donors and 162 pancreatic cancer patients from 3 independent cohorts with 74, 34 and 54 patients respectively for cohort 1, 2 and 3. It's important to note that all PAP/REG3A measurement on biological fluids were done using an ELISA assay (Dynabio SA, France) which is currently under use by several European countries in the newborn screening of Cystic Fibrosis. Statistical analysis revealed a significant increase of PAP/REG3A level in all PDA′ patients cohort (FIG. 1A, P<0.001 or P<0.0001). An optimal cut-off for PAP/REG3A level of 17.5 μg/L was identified and patients with PAP/REG3A level of 17.5 μg/L or higher had shorter overall survival (FIGS. 1B and 1C, P=0.0203 and P=0.0363 respectively). In agreement with previous data, patients with PAP/REG3A level of 17.5 μg/L or higher showed higher grade PDA at diagnosis, with an increase of stage 3 PDA from 42% to 67% in <17.5 μg/L vs. >17.5 μg/L respectively (FIG. 1D). Percentage of patients with resectable tumor at diagnosis was also consistent with previous data as 72% of patients with 17.5 μg/L PAP/REG3A or higher were non-resectable vs. 40% for patients with low PAP/REG3A level (<17.5 μg/L) (FIG. 1E). These results indicate that high circulating PAP/REG3A level in pancreatic cancer patients predicts shorten survival and worst PDA stage at diagnosis.

PAP/REG3A Enhances PDA Cancer Cell Aggressiveness

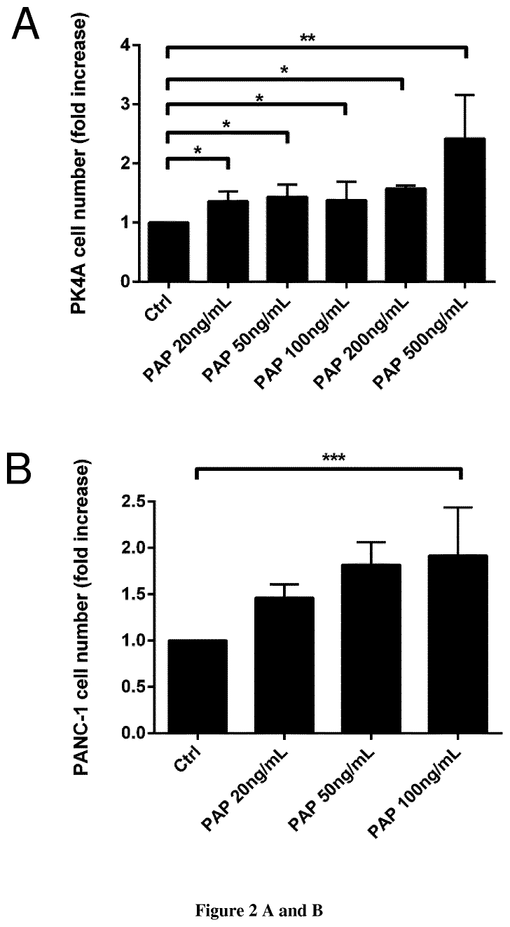

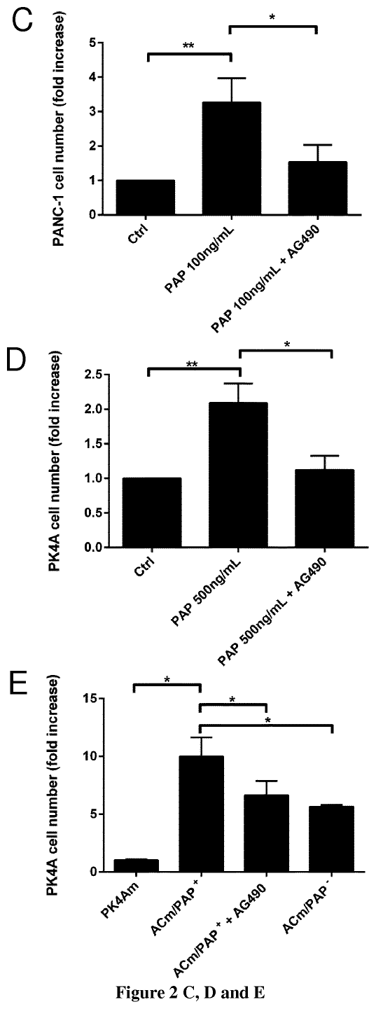

In order to determine the influence of circulating PAP/REG3A on PDA and patients' survival, and regarding our hypothesis of PAP/REG3A role on nervous system alterations and PNI, we analyzed its impact on tumor cells motility, by using specific mouse PAP/REG3β (FIG. 2A) or human PAP/REG3A (FIG. 2B) recombinant proteins. As suspected, both proteins enhanced respectively mouse (Pk4A, FIG. 2A, P<0.05 and P<0.01) and human (Panc-1, FIG. 2B, P<0.01 and P<0.001) pancreatic cancer cell migration in a dose-dependent manner. Then, we confirmed whether such induction of cancer cell migratory ability was dependent on intra-cellular signaling activated specifically by PAP/REG3A. While little is known about specific PAP/REG3A receptor, the required activation of STAT3/AKT following PAP/REG3A stimulation has been reported [29,30]. Using AG490, a STAT3 specific inhibitor, we could impair PAP/REG3A effect on Panc-1 migration (FIG. 2C, P<0.05). We further evaluated then revealed that pancreatic cancer cells, in presence of PAP/REG3β, exhibited increased invasive abilities (FIG. 2D, P<0.01), also inhibited by AG490 treatments (FIG. 2D, P<0.05). As we previously demonstrated that PAP/REG3A is mainly produced by pancreatic acinar cells (Data not shown), we confirmed above data using acinar cell conditioned media (ACm) rather than PAP/REG3A recombinant protein. We extracted and cultured acinar cells from healthy mice then retrieved their conditioned media that we depleted in PAP/REG3β (ACm/PAP−) or not (ACm/PAP+). As depicted in FIG. 2E, ACm/PAP+media enhanced Pk4A migratory abilities by 10-fold (P<0.05). Such increase is significantly reduced either by AG490 treatment (P<0.05) or following incubation with ACm/PAP−, the PAP/REG3β-depleted media (P<0.05) (FIG. 2E). These data show a significant role of PAP/REG3A/REG3β in migration and invasion abilities of pancreatic cancer cells, through JAK2/STAT3 signaling activation.

PAP/REG3A Increases Peri-Neural Invasion (PNI)

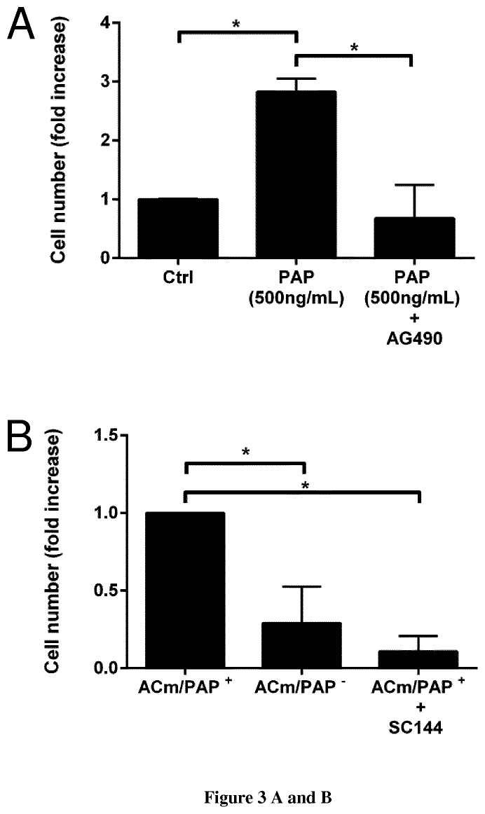

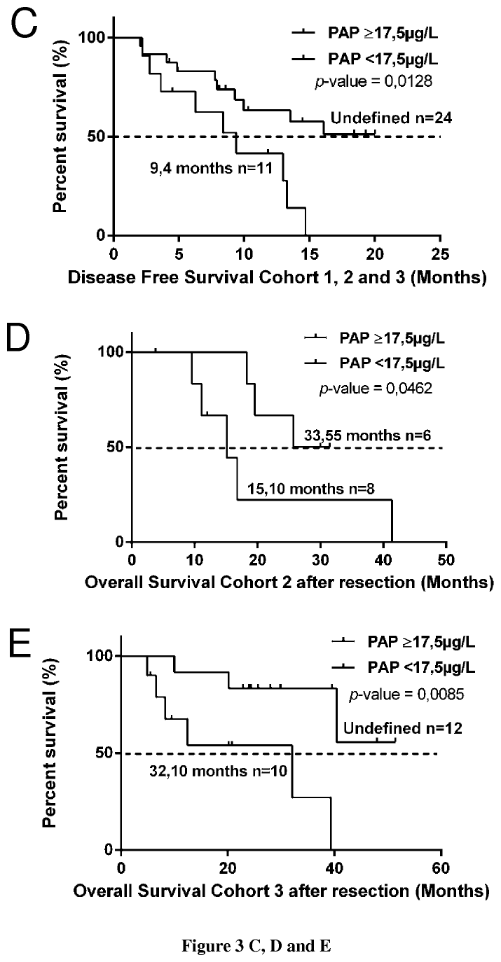

Using human PDA slides, we observed that PAP/REG3A staining is associated with nerve density (Data not shown). Indeed, nerve fibers are mainly present in tissues areas where PAP/REG3A is strongly expressed. Moreover, immunostaining done on human PDA slides showed that tumor cells present within PNI events are expressing gp130 and, therefore, could respond to PAP/REG3A induced signaling (Data not shown). To further deepen the suspected role of PAP/REG3A as favoring PNI, we designed a specific ex-vivo experiment. Such experiment was done using mouse sciatic nerve fibers that we incubated with Pk4A cells+/−PAP/REG3β mouse recombinant protein (Data not shown). After 72 hours of co-culture, nerve fibers were fixed and paraffin-embedded sections prepared to determine, by immunohistological staining, the presence of Pk4A cells that have invaded and migrated within nerve fibers. First, we observed that the presence of PAP/REG3β in the co-culture nerve/Pk4A led to a 2.9-fold increase in tumor cell number within nerve sections (P<0.05, FIG. 3A). As for previous experiments (FIGS. 2C to E), the use of the STAT3 inhibitor AG490 impairs significantly the effect of PAP/REG3β on nerve fibers invasion by pancreatic cancer cells (P>0.05, FIG. 3A). We confirmed the role of PAP/REG3β on PNI using acinar cell media containing or depleted in PAP/REG3β. Indeed, the PNI obtained following Pk4A incubation with nerve fibers and acinar cell media containing PAP/REG3β (ACm/PAP+) was drastically reduced when using PAP/REG3β-depleted acinar cell media (ACm/PAP−) (P<0.05, FIG. 3B) or following treatment of Pk4A cells by SC144 (a gp130 inhibitor, [31]), impairing PAP/REG33 stimulation (P<0.05, FIG. 3B). These results confirm the direct impact of PAP/REG3β on PNI, in a JAK2/STAT3-dependent manner.

High PAP/REG3A Levels are Associated with Worst Prognosis for Resected Patients

PNI is at present considered as one of the major cause of tumor-relapse for patients following PDA resection [32] and so impacting their overall survival. Considering our previous data which revealed that PAP/REG33 enhanced PNI but also our data revealing that PAP/REG3A detection in patients' sera is associated with worse prognosis, we wondered if measurement of PAP/REG3A in serum from pancreatic cancer resected patients is also associated with PNI related parameters. Supporting our hypothesis, we observed that disease-free survival was significantly reduced for patients with PAP/REG3A level of 17.5 μg/L or higher compared to patients with PAP/REG3A level lower than 17.5 μg/L (P=0.0128, FIG. 3C). Moreover, we correlated this result to overall survival of PDAs' patients following surgical resection of their pancreatic tumor. Using the same optimal PAP/REG3A cut-off value of 17.5 μg/L, we revealed, in 2 different cohorts, that patients with PAP/REG3A value higher than 17.5 μg/L had a shorter survival following PDA resection (P=0.0462, FIG. 3D and P=0.0085, FIG. 3E). Altogether, those data confirm the clinical association between PAP/REG3A level in serum and worse prognosis, even for PDA resected patients, suggesting the implication of PAP/REG3A in PDA relapse.

### DISCUSSION

The management of PDA is limited by the lack of accurate therapies. Besides, clinicians are in urgent need of efficient disease biomarkers allowing patients stratification on the basis of their response to treatment as well as to their clinicopathological features or PDA-associated phenotypes, both impacting clinical cares and survival. In the present study we demonstrated that peri-tumoral microenvironment, through the secretion of PAP/REG3A by inflamed acinar cells, increases cancer cell aggressiveness and favors peri-neural dissemination.

First, we reported that inflamed acinar cells from peri-tumoral (PT) compartment are the original source of PAP/REG3A expression in PDA. Regarding our starting hypothesis on the influence of PAP/REG3A on PNI in PDA, it's important to note that those PAP/REG3A expressing areas are the one rich in nerve fibers. This correlation supports our original concept as tumor cells and nerve fibers, which are in those areas, can respond to PAP/REG3A driven signaling as we showed they both express gp130. The association of PAP/REG3A staining and nerve fibers in similar areas could also lead to a new hypothesis that PAP/REG3A favors neural remodeling. Deepening this assumption could bring new insights on mechanisms associated to neural remodeling in PDA, since its early lesions.

On the side of PNI, numerous factors from several cell types within PDA tumor mass were determined as impacting PNI. Indeed, cancer cells, macrophages and CAFs were already determined as fostering PNI [33-35] or NR [15]. Interestingly, our study revealed the concrete influence of peri-tumoral areas on PNI. Our data, by showing such an impacting role from PT areas, suggest that deepened studies should be conducted in order to screen more in details the various mode of action of inflamed acinar cells onto PNI or NR. Beyond the numerous secreted proteins already associated to PNI, extra-cellular vesicles, which were reported as an important CAFs-mediated support to tumor cells [5] or free circulating miRNA [36] are two major inter-cellular communication modes that should be explored in the connection inflamed acinar cells/PNI. Indeed, while CAFs are the PDA official “factories” for secreted proteins, that impact neighboring and more distant stromal or tumoral cells [37] our data shed some light on an under estimated compartment combining both inflamed acinar cells and infiltrating immune cells. Determining the inflamed acinar cells impacts to its neighboring tumor cells and their relative activity within PDA carcinogenesis, and associated phenotype, should constitute important keys for deciphering new clinical tools. In this direction, the evaluation of PAP/REG3A level in PDA patients' sera constitutes a proof of concept as well as an interesting promise for patients' stratification.

Also, we highlighted that PAP/REG3A, is able to enhance pancreatic tumor cells aggressiveness through the activation of STAT3 proteins which is known to govern several key oncogenic signaling pathways [38]. Recently, Wormainn and colleagues reported that persistent activation of STAT3 is involved in the progression of PDA and was associated with p53 mutation in tumor cells [39]. Consistent with data obtained from Worminn's study, most of pancreatic cancer cells have constitutively activated STAT3 [40] as well as mutated p53 [41]. However, while constitutive/persistent STAT3 activation was showed in absence of active p53 [39], numerous studies reported that p53-mutated pancreatic cancer cells were still able to respond then enhance STAT3 signaling [42]. Interestingly, we showed in our study that PAP/REG3A is able to enhance migration and invasion ability of p53-mutated pancreatic tumor cells, through a STAT3-dependent mechanism. This data confirmed that even if STAT3 is constitutively/persistently activated in PDA tumor cells, STAT3 is still able to respond then drive PAP/REG3A stimulation. Therapeutic strategy using Stat3/Jak2 inhibitors was already reported as reducing tumor growth and chemoresistance [43] as well as impacting desmoplastic stromal reaction [44]. Those data reinforce the need to deeply understand the various processes that activate STAT3 signaling or STAT3 downstream effectors in PDA. Indeed, determining STAT3 bypass, which render Stat3/Jak2 inhibitors inefficient, could highlight potent STAT3-associated targets for treatment of PDA.

In our study, we revealed that PAP/REG3A favors peri-neural invasion, a well-known associated factor of pancreatic tumor cell dissemination and tumor recurrence [9]. While recent improvements on molecules or mechanisms associated to PNI were reported [45,46], the importance of such phenomenon on PDAs' patients and its possible use as therapeutic target is from far insufficient. In our model, the PAP/REG3A-favored PNI is dependent on STAT3 activation which strengthens the interest of Stat3-targeting therapy for PDA as well as for pancreatic cancer associated PNI [47]. Indeed, STAT3-inhibitors as well as PAP/REG3A blocking antibody treatments should be investigated for patients that underwent PDA surgical resection in order to determine the impact of those treatments on tumor recurrence and disease-free survival.

In our study, blood PAP/REG3A levels were not significantly associated with patients' age, gender or TNM staging. Interestingly, when we stratified PDA patients based on their eligibility to surgery, we found that resected PDA patients with a high level of PAP/REG3A had a significantly shorter survival than patients with a low level of PAP/REG3A. Moreover, the disease-free survival, reflecting the time before the disease recurrence, is also shorter for PDA patients with a high PAP/REG3A level compared to patients with a low PAP/REG3A level. Regarding those data, and taking into account that level of PAP/REG3A found in sera from PDA patients comes from peri-tumoral acinar cells, it is not unreasonable to suppose that PAP/REG3A blood levels in PDA can reflect primary tumor aggressiveness or dimensions which are finally related to clinical outcome of patients. Regarding the crucial needs of clinicians in terms of diagnosis of PDA patients as in terms of specific therapies, the discovery of new efficient biomarkers fostering the prognosis and the stratification of PDA patients is a meaningful goal to reach as far as possible. While immune biomarkers [48] and miRNAs [49] focused recent discoveries in the field [50], our data on PAP/REG3A highlights the underestimated field of peri-tumoral components as a potent source of efficient biomarkers.

### CONCLUSIONS

In summary, our results demonstrated the influence of PAP/REG3A on PDA-associated PNI, deepened our knowledges on the impact of peri-tumoral compartment as well as STAT3 signaling implication in PDA carcinogenesis, and may open new therapeutic fields. Also, it is tempting to suggest that high levels of PAP/REG3A in blood, may serve as a promising tool for PDA patients' stratification, thereby favoring a targeted and individualized therapy in order to prevent tumor relapse and local dissemination through NR or PNI.

### REFERENCES

Throughout this application, various references describe the state of the art to which this invention pertains. The disclosures of these references are hereby incorporated by reference into the present disclosure.
1. Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res. 1 juin 2014; 74(11):2913-21.
2. Ryan D P, Hong T S, Bardeesy N. Pancreatic adenocarcinoma. N Engl J Med. 27 Nov. 2014; 371(22):2140-1.
3. Von Hoff D D, Ervin T, Arena F P, Chiorean E G, Infante J, Moore M, Seay T, Tjulandin S A, Ma W W, Saleh M N, Harris M, Reni M, Dowden S, Laheru D, Bahary N, Ramanathan R K, Tabemero J, Hidalgo M, Goldstein D, Van Cutsem E, Wei X, Iglesias J, Renschler M F. Increased survival in pancreatic cancer with nab-paclitaxel plus gemcitabine. N Engl J Med. 31 Oct. 2013; 369(18):1691-703.
4. Zhao X, Fan W, Xu Z, Chen H, He Y, Yang G, Yang G, Hu H, Tang S, Wang P, Zhang Z, Xu P, Yu M. Inhibiting tumor necrosis factor-alpha diminishes desmoplasia and inflammation to overcome chemoresistance in pancreatic ductal adenocarcinoma. Oncotarget. 8 Nov. 2016;
5. Leca J, Martinez S, Lac S, Nigri J, Secq V, Rubis M, Bressy C, Sergé A, Lavaut M-N, Dusetti N, Loncle C, Roques J, Pietrasz D, Bousquet C, Garcia S, Granjeaud S, Ouaissi M, Bachet J B, Brun C, lovanna J L, Zimmermann P, Vasseur S, Tomasini R. Cancer-associated fibroblast-derived annexin A6+ extracellular vesicles support pancreatic cancer aggressiveness. J Clin Invest. 1 Nov. 2016; 126(11):4140-56.
6. Flint T R, Janowitz T, Connell C M, Roberts E W, Denton A E, Coll A P, Jodrell D I, Fearon D T. Tumor-Induced IL-6 Reprograms Host Metabolism to Suppress Anti-tumor Immunity. Cell Metab. 8 Nov. 2016; 24(5):672-84.
7. Mayers J R, Torrence M E, Danai L V, Papagiannakopoulos T, Davidson S M, Bauer M R, Lau A N, Ji B W, Dixit P D, Hosios A M, Muir A, Chin C R, Freinkman E, Jacks T, Wolpin B M, Vitkup D, Vander Heiden M G. Tissue of origin dictates branched-chain amino acid metabolism in mutant Kras-driven cancers. Science. 9 Sep. 2016; 353(6304):1161-5.
8. Demir I E, Friess H, Ceyhan G O. Neural plasticity in pancreatitis and pancreatic cancer. Nat Rev Gastroenterol Hepatol. November 2015; 12(11):649-59.
9. Liang D, Shi S, Xu J, Zhang B, Qin Y, Ji S, Xu W, Liu J, Liu L, Liu C, Long J, Ni Q, Yu X. New insights into perineural invasion of pancreatic cancer: More than pain. Biochim Biophys Acta. avr 2016; 1865(2):111-22.
10. Saloman J L, Albers K M, Li D, Hartman D J, Crawford H C, Muha E A, Rhim A D, Davis B M. Ablation of sensory neurons in a genetic model of pancreatic ductal adenocarcinoma slows initiation and progression of cancer. Proc Natl Acad Sci USA. 15 mars 2016; 113(11):3078-83.
11. Nielsen M F B, Mortensen M B, Detlefsen S. Key players in pancreatic cancer-stroma interaction: Cancer-associated fibroblasts, endothelial and inflammatory cells. World J Gastroenterol. 7 mars 2016; 22(9):2678-700.
12. Kimbara S, Kondo S. Immune checkpoint and inflammation as therapeutic targets in pancreatic carcinoma. World J Gastroenterol. 7 Sep. 2016; 22(33):7440-52.
13. Mei L, Du W, Ma W W. Targeting stromal microenvironment in pancreatic ductal adenocarcinoma: controversies and promises. J Gastrointest Oncol. juin 2016; 7(3):487-94.
14. Chalabi-Dchar M, Cassant-Sourdy S, Duluc C, Fanjul M, Lulka H, Samain R, Roche C, Breibach F, Delisle M-B, Poupot M, Dufresne M, Shimaoka T, Yonehara S, Mathonnet M, Pyronnet S, Bousquet C. Loss of Somatostatin Receptor Subtype 2 Promotes Growth of KRAS-Induced Pancreatic Tumors in Mice by Activating PI3K Signaling and Overexpression of CXCL16. Gastroenterology. juin 2015; 148(7):1452-65.
15. Secq V, Leca J, Bressy C, Guillaumond F, Skrobuk P, Nigri J, Lac S, Lavaut M-N, Bui T-T, Thakur A K, Callizot N, Steinschneider R, Berthezene P, Dusetti N, Ouaissi M, Moutardier V, Calvo E, Bousquet C, Garcia S, Bidaut G, Vasseur S, lovanna J L, Tomasini R. Stromal SLIT2 impacts on pancreatic cancer-associated neural remodeling. Cell Death Dis. 2015; 6:e1592.
16. Delitto D, Black B S, Sorenson H L, Knowlton A E, Thomas R M, Sarosi G A, Moldawer L L, Behrns K E, Liu C, George T J, Trevino J G, Wallet S M, Hughes S J. The inflammatory milieu within the pancreatic cancer microenvironment correlates with clinicopathologic parameters, chemoresistance and survival. BMC Cancer. 24 Oct. 2015; 15:783.
17. Karakhanova S, Link J, Heinrich M, Shevchenko I, Yang Y, Hassenpflug M, Bunge H, von Ahn K, Brecht R, Mathes A, Maier C, Umansky V, Werner J, Bazhin A V. Characterization of myeloid leukocytes and soluble mediators in pancreatic cancer: importance of myeloid-derived suppressor cells. Oncoimmunology. avr 2015; 4(4):e998519.
18. Toste P A, Nguyen A H, Kadera B E, Duong M, Wu N, Gawlas I, Tran L M, Bikhchandani M, Li L, Patel S G, Dawson D W, Donahue T R. Chemotherapy-Induced Inflammatory Gene Signature and Protumorigenic Phenotype in Pancreatic CAFs via Stress-Associated MAPK. Mol Cancer Res MCR. mai 2016; 14(5):437-47.
19. Keim V, lovanna J L, Rohr G, Usadel K H, Dagorn J C. Characterization of a rat pancreatic secretory protein associated with pancreatitis. Gastroenterology. mars 1991; 100(3):775-82.
20. Keim V, lovanna J L, Orelle B, Verdier J M, Busing M, Hopt U, Dagom J C. A novel exocrine protein associated with pancreas transplantation in humans. Gastroenterology. juill 1992; 103(1):248-54.
21. Barthellemy S, Maurin N, Roussey M, Férec C, Murolo S, Berthézène P, lovanna J L, Dagom J C, Sarles J. [Evaluation of 47,213 infants in neonatal screening for cystic fibrosis, using pancreatitis-associated protein and immunoreactive trypsinogen assays]. Arch Pediatr Organe Off Soc Francaise Pediatr. mars 2001; 8(3):275-81.
22. Gironella M, Calvo C, Femindez A, Closa D, lovanna J L, Rosello-Catafau J, Folch-Puy E. Reg3β deficiency impairs pancreatic tumor growth by skewing macrophage polarization. Cancer Res. 15 Sep. 2013; 73(18):5682-94.
23. Liu X, Wang J, Wang H, Yin G, Liu Y, Lei X, Xiang M. REG3A accelerates pancreatic cancer cell growth under IL-6-associated inflammatory condition: Involvement of a REG3A-JAK2/STAT3 positive feedback loop. Cancer Lett. 28 juin 2015; 362(1):45-60.
24. Rosty C, Christa L, Kuzdzal S, Baldwin W M, Zahurak M L, Camot F, Chan D W, Canto M, Lillemoe K D, Cameron J L, Yeo C J, Hruban R H, Goggins M. Identification of hepatocarcinoma-intestine-pancreas/pancreatitis-associated protein I as a biomarker for pancreatic ductal adenocarcinoma by protein biochip technology. Cancer Res. 15 mars 2002; 62(6):1868-75.
25. Nishimune H, Vasseur S, Wiese S, Birling M C, Holtmann B, Sendtner M, lovanna J L, Henderson C E. Reg-2 is a motoneuron neurotrophic factor and a signalling intermediate in the CNTF survival pathway. Nat Cell Biol. déc 2000; 2(12):906-14.
26. Haldipur P, Dupuis N, Degos V, Moniaux N, Chhor V, Rasika S, Schwendimann L, le Charpentier T, Rougier E, Amouyal P, Amouyal G, Dournaud P, Bréchot C, El Ghouzzi V, Faivre J, Fleiss B, Mani S, Gressens P. HIP/PAP prevents excitotoxic neuronal death and promotes plasticity. Ann Clin Transl Neurol. October 2014; 1(10):739-54.
27. Guillaumond F, Leca J, Olivares O, Lavaut M-N, Vidal N, Berthezene P, Dusetti N J, Loncle C, Calvo E, Turrini O, lovanna J L, Tomasini R, Vasseur S. Strengthened glycolysis under hypoxia supports tumor symbiosis and hexosamine biosynthesis in pancreatic adenocarcinoma. Proc Natl Acad Sci USA. 5 mars 2013; 110(10):3919-24.
28. Gout J, Pommier R M, Vincent D F, Ripoche D, Goddard-Léon S, Colombe A, Treilleux I, Valcourt U, Tomasini R, Dufresne M, Bertolino P, Bartholin L. The conditional expression of KRAS G12D in mouse pancreas induces disorganization of endocrine islets prior the onset of ductal pre-cancerous lesions. Pancreatol Off J Int Assoc Pancreatol IAP Al. juin 2013; 13(3):191-5.
29. Closa D, Motoo Y, lovanna J L. Pancreatitis-associated protein: from a lectin to an anti-inflammatory cytokine. World J Gastroenterol. 14 janv 2007; 13(2):170-4.
30. Folch-Puy E, Granell S, Dagorn J C, lovanna J L, Closa D. Pancreatitis-associated protein I suppresses N F-kappa B activation through a JAK/STAT-mediated mechanism in epithelial cells. J Immunol Baltim Md. 1950. 15 mars 2006; 176(6):3774-9.
31. Xu S, Grande F, Garofalo A, Neamati N. Discovery of a novel orally active small-molecule gp130 inhibitor for the treatment of ovarian cancer. Mol Cancer Ther. juin 2013; 12(6):937-49.
32. Shimada K, Nara S, Esaki M, Sakamoto Y, Kosuge T, Hiraoka N. Intrapancreatic nerve invasion as a predictor for recurrence after pancreaticoduodenectomy in patients with invasive ductal carcinoma of the pancreas. Pancreas. avr 2011; 40(3):464-8.
33. Hibi T, Mori T, Fukuma M, Yamazaki K, Hashiguchi A, Yamada T, Tanabe M, Aiura K, Kawakami T, Ogiwara A, Kosuge T, Kitajima M, Kitagawa Y, Sakamoto M. Synuclein-gamma is closely involved in perineural invasion and distant metastasis in mouse models and is a novel prognostic factor in pancreatic cancer. Clin Cancer Res Off J Am Assoc Cancer Res. 15 avr 2009; 15(8):2864-71.
34. Cavel O, Shomron O, Shabtay A, Vital J, Trejo-Leider L, Weizman N, Krelin Y, Fong Y, Wong R J, Amit M, Gil Z. Endoneurial macrophages induce perineural invasion of pancreatic cancer cells by secretion of GDNF and activation of RET tyrosine kinase receptor. Cancer Res. 15 Nov. 2012; 72(22):5733-43.
35. Gao Z, Wang X, Wu K, Zhao Y, Hu G. Pancreatic stellate cells increase the invasion of human pancreatic cancer cells through the stromal cell-derived factor-1/CXCR4 axis. Pancreatol Off J Int Assoc Pancreatol IAP Al. 2010; 10(2-3):186-93.
36. Sun T, Kong X, Du Y, Li Z. Aberrant MicroRNAs in Pancreatic Cancer: Researches and Clinical Implications. Gastroenterol Res Pract [Internet]. 2014 [cité 15 Nov. 2016]; 2014. Disponible sur: http://www.ncbi.nlm.nih.gov/pmc/articles/PMC4034662/
37. Pan B, Liao Q, Niu Z, Zhou L, Zhao Y. Cancer-associated fibroblasts in pancreatic adenocarcinoma. Future Oncol Lond Engl. September 2015; 11(18):2603-10.
38. Corcoran R B, Contino G, Deshpande V, Tzatsos A, Conrad C, Benes C H, Levy D E, Settleman J, Engelman J A, Bardeesy N. STAT3 plays a critical role in KRAS-induced pancreatic tumorigenesis. Cancer Res. 15 juill 2011; 71(14):5020-9.
39. Wörmann S M, Song L, Ai J, Diakopoulos K N, Kurkowski M U, Görgülü K, Ruess D, Campbell A, Doglioni C, Jodrell D, Neesse A, Demir I E, Karpathaki A-P, Barenboim M, Hagemann T, Rose-John S, Sansom O, Schmid R M, Protti M P, Lesina M, Algiil H. Loss of P53 Function Activates JAK2-STAT3 Signaling to Promote Pancreatic Tumor Growth, Stroma Modification, and Gemcitabine Resistance in Mice and Is Associated With Patient Survival. Gastroenterology. juill 2016; 151(1):180-193.e12.
40. Wei D, Le X, Zheng L, Wang L, Frey J A, Gao A C, Peng Z, Huang S, Xiong H Q, Abbruzzese J L, Xie K. Stat3 activation regulates the expression of vascular endothelial growth factor and human pancreatic cancer angiogenesis and metastasis. Oncogene. 23 janv 2003; 22(3):319-29.
41. Deer E L, Gonzilez-Hernindez J, Coursen J D, Shea J E, Ngatia J, Scaife C L, Firpo M A, Mulvihill S J. Phenotype and genotype of pancreatic cancer cell lines. Pancreas. mai 2010; 39(4):425-35.
42. Huang X-Y, Huang Z-L, Xu B, Chen Z, Re T J, Zheng Q, Tang Z-Y, Huang X-Y. Erratum to: Elevated MTSS1 expression associated with metastasis and poor prognosis of residual hepatitis B-related hepatocellular carcinoma. J Exp Clin Cancer Res CR. 24 juin 2016; 35(1):102.
43. Ioannou N, Seddon A M, Dalgleish A, Mackintosh D, Solca F, Modjtahedi H. Acquired resistance of pancreatic cancer cells to treatment with gemcitabine and HER-inhibitors is accompanied by increased sensitivity to STAT3 inhibition. Int J Oncol. mars 2016; 48(3):908-18.
44. Cowan R W, Maitra A, Rhim A D. A New Scalpel for the Treatment of Pancreatic Cancer: Targeting Stromal-Derived STAT3 Signaling. Gastroenterology. déc 2015; 149(7):1685-8.
45. Bapat A A, Munoz R M, Von Hoff D D, Han H. Blocking Nerve Growth Factor Signaling Reduces the Neural Invasion Potential of Pancreatic Cancer Cells. PloS One. 2016; 11(10):e0165586.
46. Nomura A, Majumder K, Giri B, Dauer P, Dudeja V, Roy S, Banerjee S, Saluja A K. Inhibition of N F-kappa B pathway leads to deregulation of epithelial-mesenchymal transition and neural invasion in pancreatic cancer. Lab Investig J Tech Methods Pathol. 24 Oct. 2016;
47. Guo K, Ma Q, Li J, Wang Z, Shan T, Li W, Xu Q, Xie K. Interaction of the sympathetic nerve with pancreatic cancer cells promotes perineural invasion through the activation of STAT3 signaling. Mol Cancer Ther. mars 2013; 12(3):264-73.
48. Connor A A, Denroche R E, Jang G H, Timms L, Kalimuthu S N, Selander I, McPherson T, Wilson G W, Chan-Seng-Yue M A, Borozan I, Ferretti V, Grant R C, Lungu I M, Costello E, Greenhalf W, Palmer D, Ghaneh P, Neoptolemos J P, Buchler M, Petersen G, Thayer S, Hollingsworth M A, Sherker A, Durocher D, Dhani N, Hedley D, Serra S, Pollett A, Roehrl M H A, Bavi P, Bartlett J M S, Cleary S, Wilson J M, Alexandrov L B, Moore M, Wouters B G, McPherson J D, Notta F, Stein L D, Gallinger S. Association of Distinct Mutational Signatures With Correlates of Increased Immune Activity in Pancreatic Ductal Adenocarcinoma. JAMA Oncol. 20 Oct. 2016;
49. Vila-Navarro E, Vila-Casadesus M, Moreira L, Duran-Sanchon S, Sinha R, Ginés À, Fernindez-Esparrach G, Miquel R, Cuatrecasas M, Castells A, Lozano J J, Gironella M. MicroRNAs for Detection of Pancreatic Neoplasia: Biomarker Discovery by Next-generation Sequencing and Validation in 2 Independent Cohorts. Ann Surg. 26 mai 2016;
50. Karandish F, Mallik S. Biomarkers and Targeted Therapy in Pancreatic Cancer. Biomark Cancer. 2016; 8 (Suppl 1):27-35.
51. Bapat A A, Hostetter G, Von Hoff D D, Han H. Perineural invasion and associated pain in pancreatic cancer. Nature reviews Cancer 2011; 11(10):695-707.
52. Ceyhan G O, Bergmann F, Kadihasanoglu M, Altintas B, Demir I E, Hinz U, et al. Pancreatic neuropathy and neuropathic pain—a comprehensive pathomorphological study of 546 cases. Gastroenterology 2009; 136(1):177-86 el.
53. Liu X, Wang J, Wang H, Yin G, Liu Y, Lei X, Xiang M. REG3A accelerates pancreatic cancer cell growth under IL-6-associated inflammatory condition: Involvement of a REG3A-JAK2/STAT3 positive feedback loop. Cancer Lett. 2015 Jun. 28; 362(1):45-60.
54. Secq V, Leca J, Bressy C, Guillaumond F, Skrobuk P, Nigri J, et al. Stromal SLIT2 impacts on pancreatic cancer-associated neural remodeling. Cell death & disease 2015; 6:e1592.
55. Stopczynski R E, Normolle D P, Hartman D J, Ying H, DeBerry J J, Bielefeldt K, et al. Neuroplastic changes occur early in the development of pancreatic ductal adenocarcinoma. Cancer research 2014; 74(6): 1718-27.
56. Wang J, Zhou H, Han Y, Liu X, Wang M, Wang X, Yin G, Li X, Xiang M. SOCS3 methylation in synergy with Reg3A overexpression promotes cell growth in pancreatic cancer. J Mol Med (Berl). 2014 December; 92(12):1257-69.
57. Ye Y, Xiao L, Wang S J, Yue W, Yin Q S, Sun M Y, Xia W, Shao Z Y, Zhang H. Up-regulation of REG3A in colorectal cancer cells confers proliferation and correlates with colorectal cancer risk. Oncotarget. 2016 Jan. 26; 7(4):3921-33.

1. A method for predicting the survival time of a patient suffering from pancreatic ductal adenocarcinoma (PDA) comprising the steps of: i) determining the expression level of REG3A in a biological sample obtained from the patient, ii) comparing the expression level determined at step i) with a predetermined reference value and iii) concluding that the patient will have a long survival time when the level determined at step i) is lower than the predetermined reference value or concluding that the patient will have a short survival time when the level determined at step i) is equal or higher than the predetermined reference value.

2. A method for predicting the disease-free survival (DFS) of a PDA resected patient comprising the steps of: i) determining the expression level of REG3A in a biological sample obtained from the patient, ii) comparing the expression level determined at step i) with a predetermined reference value and iii) concluding that the patient will have a long disease-free survival when the level determined at step i) is lower than the predetermined reference value or concluding that the patient will have a short disease-free survival when the level determined at step i) is equal or higher than the predetermined reference value.

3. A method of determining whether the pancreatic ductal adenocarcinoma (PDA) of a patient is a low risk tumor or a high risk tumor, comprising the steps of: (i) determining the expression level of REG3A in a biological sample obtained from the patient, (ii) comparing the expression level of REG3A in the biological sample with a predetermined reference value, and (iii) concluding that the pancreatic ductal adenocarcinoma (PDA) of the patient is a low risk tumor when the level determined at step i) is lower than the predetermined reference value or concluding that pancreatic ductal adenocarcinoma (PDA) of the patient is a high risk tumor when the level determined at step i) is equal or higher than the predetermined reference value.

4. (canceled)

5. The method according to claim 11 wherein said REG3A inhibitor is selected from the group consisting of a small organic molecule, a polypeptide, an aptamer, an antibody, an oligonucleotide and a ribozyme.

6. The method according to claim 11 wherein said REG3A inhibitor is a REG3A expression inhibitor, a gp130 antagonist or a JAK2/STAT3 signaling inhibitor.

7. The method according to claim 11 wherein said REG3A inhibitor is SC144 or AG490.

8. The method according to claim 11, wherein the REG3A inhibitor is administered in combination with an anti-PDA treatment.

9. The method according to claim 8, wherein said anti-PDA treatment is selected from the group consisting of gemcitabine, fluorouracil, FOLFIRINOX (fluorouracil, irinotecan, oxaliplatin, and leucovorin), nab-paclitaxel, inhibitors of programmed death 1 (PD-1), anti-CLA4 antibodies, EGFR inhibitors, chemoradiotherapy, inhibitors of PARP, inhibitors of Sonic Hedgehog, gene therapy and radiotherapy.

10. A method of screening a candidate compound for use as a drug for treating PDA in a patient in need thereof, wherein the method comprises the steps of:
providing a REG3A or a cell, tissue sample or organism expressing a REG3A,
providing a candidate compound
measuring activity of the REG3A in the presence of the candidate compound, and
selecting positively candidate compounds that inhibit the REG3A activity.

11. A method of treating high risk pancreatic ductal adenocarcinoma (PDA) in a patient in need thereof comprising the steps of: i) determining whether the pancreatic ductal adenocarcinoma (PDA) of a patient is a high risk tumor by performing the method according to claim 3, and ii) administering a REG3A inhibitor to the patient when said patient is identified as having a high risk tumor.

12. The method according to claim 6, wherein the JAK2/STAT3 signaling inhibitor is a JAK2 inhibitor or a STAT3 antagonist.

13. The method according to claim 9, wherein said EGFR inhibitor is erlotinib.

14. The method according to claim 9, wherein said inhibitor of PD-1 is PD-1 ligand (PD-L1).

15. The method of claim 10, wherein the candidate compound is a small organic molecule, a polypeptide, an aptamer, an antibody or an intra-antibody.
